(19) United States
(12) Patent Application Publication
(10) Pub. No.: US 2025/0255468 A1
(43) Pub. Date: Aug. 14, 2025

(54) SYSTEMS, DEVICES, AND RELATED METHODS FOR COUPLING MEDICAL DEVICES

(71) Applicant: Boston Scientific Scimed, Inc., Maple Grove, MN (US)

(72) Inventors: Gabrielle GRAMZA, Mishawaka, IN (US); Daniel L. EADS, Center Point, IN (US); Bradley Randall BLACK, Bloomington, IN (US); Camron HAGEMEYER, Bloomington, IN (US); Thomas M. RAGONESE, Bloomington, IN (US)

(73) Assignee: Boston Scientific Scimed, Inc., Maple Grove, MN (US)

(21) Appl. No.: 19/048,245

(22) Filed: Feb. 7, 2025

Related U.S. Application Data
(60) Provisional application No. 63/552,727, filed on Feb. 13, 2024.

(51) Int. Cl. A61B 1/018

(52) U.S. Cl. CPC A61B 1/018

(57) ABSTRACT

Systems, devices, and related methods for coupling medical devices are described. A medical system may include a first medical device having a handle and a shaft extending distally from the handle, and may include a second medical device including a handle having an actuator and a shaft extending distally from the handle. The actuator may be configured to deflect the shaft of the second medical device. The medical system may further include an attachment device that may be configured to removably couple the first medical device to the second medical device. The attachment device may include a linkage that may be configured to be coupled to the actuator. The linkage may be external to the handle of the second medical device. The linkage may include a lever and movement of the lever may be configured to articulate the actuator of the second medical device.

### CROSS-REFERENCE TO RELATED APPLICATIONS

This application claims the benefit of priority to U.S. Provisional Application No. 63/552,727, filed on Feb. 13, 2024, which is incorporated by reference herein in its entirety.

### TECHNICAL FIELD

Various aspects of this disclosure relate generally to systems, devices, and methods for coupling medical devices. In particular, aspects of this disclosure relate to systems, devices, and methods for coupling medical devices for accessing a body lumen and/or delivering medical instruments to a body lumen.

### BACKGROUND

During medical procedures, such as endoscopy procedures, an operator may insert a medical device into a patient, and guide that medical device through tortuous anatomy for positioning the device at a target treatment site in the body. For example, an operator may insert a bronchoscope into lung passages to diagnose and/or treat the lungs. Accessing anatomy of small sizes, e.g., bronchioles of a lung, may present challenges to the device accessing the target treatment site. For example, a bronchoscope may have a diameter that is larger than a diameter of certain bronchioles.

### SUMMARY

Each of the aspects disclosed herein may include one or more aspects of the features described in connection with any of the other disclosed aspects.

According to some aspects of the present disclosure, a medical system may include a first medical device having a handle and a shaft extending distally from the handle. The medical system may include a second medical device including a handle having an actuator and a shaft extending distally from the handle. The actuator may be configured to deflect the shaft of the second medical device. The medical system may also include an attachment device configured to removably couple the first medical device to the second medical device. The attachment device may include a linkage that may be configured to be coupled to the actuator. The linkage may be external to the handle of the second medical device. The linkage may include a lever and movement of the lever may be configured to articulate the actuator of the second medical device.

According to some aspects, the attachment device may include a housing that may be movably coupled to one or more elements of the linkage. The attachment device may further include a channel for receiving the second medical device. In some examples, the attachment device may be removably coupled to the first medical device and the second medical device. In some examples, the attachment device may be configured such that an actuator of the first medical device and the lever of the attachment device may be actuatable by a single hand of a user. In some examples, the linkage may further include a first arm, a second arm, and a third arm movably coupled to one another. In some examples, a first end of the lever may be free and a second end of the lever may be coupled to a first end of the first arm. A second end of the first arm may be coupled to a first end of the second arm. A second end of the second arm may be coupled to a first end of the third arm. A second end of the third arm may be coupled to the actuator of the second medical device. In some examples, the third arm may have an arcuate shape. In some examples, the second end of the lever may be coupled to the first end of the first arm via a first pin which may be movable within a first slot of a housing of the attachment device. The second end of the first arm may be coupled to the first end of the second arm via a second pin which may be movable within a second slot of the housing. In some examples, the first slot and the second slot may extend parallel to one another. In some examples, the second end of the second arm may be coupled to the first end of the third arm via a hinge. In some examples, the hinge may be a living hinge. In some examples, the lever may include a contact element positioned outside of the housing of the attachment device. In some examples, the contact element may have a C or U shape. In some examples, the contact element may have a ring shape. In some examples, the linkage may further include a spring coupled to the lever and the spring may be configured to bias the lever to a relaxed state.

According to some aspects of the present disclosure, a medical system may include a first medical device having a handle coupled to a shaft and an actuator for deflecting the shaft of the first medical device. The medical system may include a second medical device having a handle, a shaft coupled to the handle, and an actuator for deflecting the shaft of the second medical device. The medical system may also include one or more attachment devices coupling the handle of the second medical device to a proximal portion of the handle of the first medical device, such that the actuator of the second medical device may be positioned on a first side of the handle of the first medical device. The actuator of the first medical device may be positioned on a second side of the handle of the first medical device. The first side may be opposite to the second side. The actuator of the second medical device and the actuator of the first medical device may be actuatable by a single hand of a user. In some examples, the one or more attachment devices may couple to a wheel of the actuator of the first medical device. In some examples, the actuator may be a lever angled towards the handle of the first medical device in a configuration in which the one or more attachment devices may couple the handle of the second medical device to the handle of the first medical device.

According to some aspects of the present disclosure, a medical system may include a first medical device having a handle coupled to a shaft and a second medical device. The second medical device may include a shaft and a handle coupled to the shaft. The handle of the second medical device may include a handle body, an actuation mechanism which may be for deflecting the shaft of the second medical device, and an attachment device that may be configured to couple the handle body of the second medical device to the handle of the first medical device. The handle of the second medical device may have a substantially “V” shape. In some examples, a distal portion of the handle body and the attachment device may extend away from a proximal portion of the handle body, and the proximal portion of the handle body may include the actuation mechanism.

### BRIEF DESCRIPTION OF DRAWINGS

The accompanying drawings, which are incorporated in and constitute a part of this application, illustrate exemplary aspects of this disclosure and together with the description, serve to explain the principles of the present disclosure.

FIG. 1 illustrates an exemplary first medical device, in accordance with some aspects of the present disclosure.

FIG. 2A illustrates an exemplary medical system including the first medical device of FIG. 1 coupled to an exemplary second medical device, in accordance with some aspects of the present disclosure.

FIG. 2B illustrates an exemplary actuation mechanism of the exemplary medical system of FIG. 2A.

FIG. 3 illustrates an exemplary medical system including the first medical device of FIG. 1 coupled to another exemplary second medical device, in accordance with some aspects of the present disclosure.

FIG. 4 illustrates an exemplary medical system including the first medical device of FIG. 1 coupled to another exemplary second medical device, in accordance with some aspects of the present disclosure.

FIG. 5 illustrates an exemplary medical system including the first medical device of FIG. 1 coupled to yet another exemplary second medical device, in accordance with some aspects of the present disclosure.

FIGS. 6A-6B illustrate an exemplary medical system including the first medical device of FIG. 1 coupled to another exemplary second medical device, in accordance with some aspects of the present disclosure.

FIG. 7 illustrates an exemplary medical system including the first medical device of FIG. 1 coupled to yet another exemplary second medical device, in accordance with some aspects of the present disclosure.

FIGS. 8A-8B illustrate an exemplary medical system including the first medical device of FIG. 1 coupled to a further exemplary second medical device, in accordance with some aspects of the present disclosure.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although bronchoscopes are referenced herein for illustration purposes, it will be appreciated that the disclosure encompasses any suitable medical device configured to allow an operator to access and view internal body anatomy of a subject (e.g., patient) and/or to deliver medical instruments or accessory devices, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, lungs, the urinary tract, or gastrointestinal tract. It will be appreciated that, unless otherwise specified, endoscopes, duodenoscopes, gastroscopes, endoscopic ultrasonography (“EUS”) scopes, colonoscopes, ureteroscopes, laparoscopes, cystoscopes, aspiration scopes, sheaths, catheters, or any other suitable delivery device or medical device may be used in connection with the features described herein.

Features of the medical systems herein may improve navigation through tortuous body anatomy (e.g., bronchioles of a lung) and/or accessibility to smaller body lumens. According to some aspects of the present disclosure, the medical system may include an accessory scope device coupled to a primary scope device (e.g., a bronchoscope). The accessory scope device may have a reduced profile, and may have a shaft that is sized and shaped to be received within a working lumen of the primary scope device. During an exemplary medical procedure, the primary scope device may be used to position the accessory scope device at or proximate a target site. The primary scope device may provide structural support to the accessory scope device as the accessory scope device is navigated to or through tortuous anatomy. The accessory scope device may include a working lumen sized and shaped to receive medical instruments of varying sizes to facilitate a medical procedure and/or to deliver suction.

Features of the medical systems herein may improve the ergonomics and/or usability of the accessory scope device and the primary scope device. For example, features of the medical systems herein may allow an operator to control or actuate both the accessory scope device and the primary scope device using a single hand, simultaneously or independently. According to some aspects of the present disclosure, the accessory scope device may be coupled to the primary scope device via an attachment device. The attachment device may be or may include, e.g., clips or straps. Other suitable examples of attachment devices that may be used to couple the accessory scope to the primary scope are described in U.S. Pat. No. 8,353,493, the entire disclosure of which is hereby incorporated by reference. The accessory scope device may have a shape complementary to the shape of the primary scope device to further facilitate coupling the accessory scope device to the primary scope device. According to some aspects of the present disclosure, the attachment device may include an actuation assembly coupled to an actuation mechanism of the accessory scope for articulating a distal end of the accessory scope.

Reference will now be made in detail to examples of the present disclosure described above and illustrated in the accompanying drawings. Wherever possible, the same reference numbers will be used throughout the drawings to refer to the same or like parts.

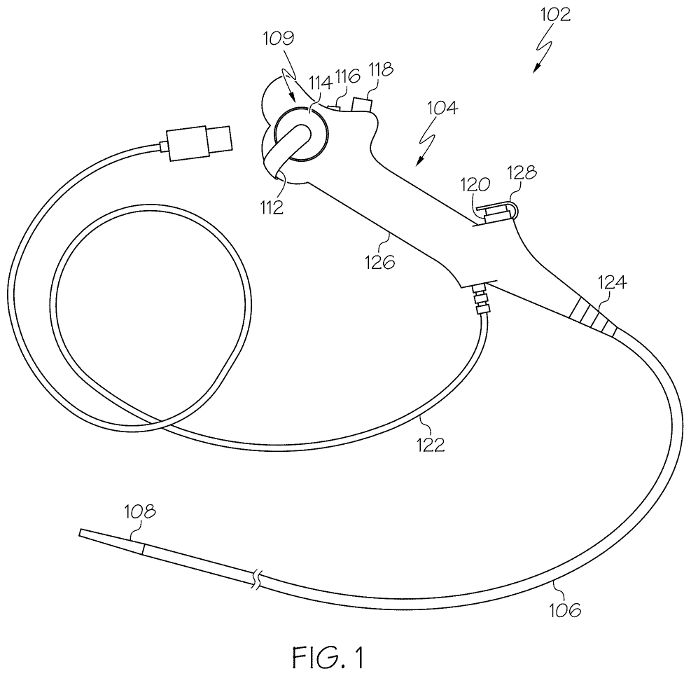

FIG. 1 illustrates an exemplary primary scope device 102 (e.g., a bronchoscope) having a handle 104 and a shaft 106 extending distally from handle 104. A stress relief portion 124 may bridge handle 104 and shaft 106. Shaft 106 may be at least partially flexible to facilitate navigation of shaft 106 through tortuous anatomical passages in the body. Shaft 106 may include a distal end portion that terminates at a distal tip 108. Distal tip 108 may include an imaging device (e.g., camera, imager, etc.), one or more light sources (e.g., LEDs, fiber optics, etc.), and an opening in communication with a working lumen extending through shaft 106 and to a port 120 on handle 104. An operator (i.e., a user) may remove a cap or seal 128 from port 120, and may insert a medical instrument or other device into port 120 and may extend the medical instrument or other device distally through the working lumen of scope device 102. Handle 104 may include a grip portion 126, which may allow an operator to grasp handle 104 during a medical procedure.

Handle 104 may include an actuation mechanism 109 having a lever 112 and a wheel 114 (e.g., a cam wheel) on handle 104 to facilitate articulation, steering, and/or deflection of shaft 106 and distal tip 108 (e.g., 180 degree articulation). Handle 104 may also include a button 116 for image capture (e.g., capture video and/or still images from the imaging device at distal tip 108) and a valve 118 to control suction, e.g., to provide air and/or water suction through the working lumen of scope device 102. Handle 104 may be coupled to an umbilicus 122. Umbilicus 122 may extend from handle 104 to one or more auxiliary devices. The one or more auxiliary devices may include a controller or control system, an imaging system, a power supply, a display, etc.

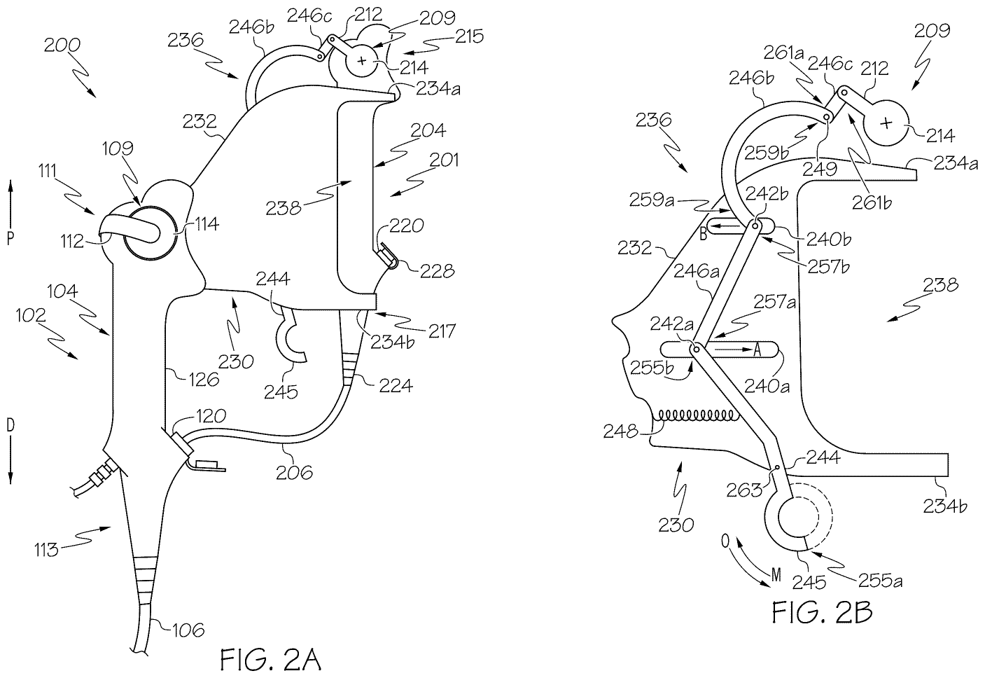

FIG. 2A illustrates a medical system 200 that includes primary scope device 102 (e.g., a first medical device) coupled an accessory scope device 201 (e.g., a second medical device). Accessory scope device 201 may include a handle 204, a shaft 206, and a stress relief portion 224. Handle 204 may include any of the features of handle 104, and shaft 206 may include any of the features of shaft 106, unless otherwise specified herein. For example, handle 204 may include an actuation mechanism 209 having a lever 212 coupled to a wheel 214 (e.g., a cam wheel), a port 220 in communication with a working lumen of shaft 206, and a cap or seal 228.

Shaft 206 of accessory scope device 201 may be sized and shaped to be received within port 120 and the working lumen of shaft 106 of primary scope device 102. For example, the working lumen of shaft 106 of primary scope device 102 may have a working lumen width of approximately 1.0 mm to approximately 2.6 mm. Shaft 206 of accessory scope device 201 may accordingly have an outer diameter/width that ranges from less than 1.0 mm to less than 2.6 mm. A length of shaft 206 may be such that shaft 206 may be passed through the working lumen of shaft 106 of primary scope device 102 and extended from an opening in distal tip 108 of primary scope device 102.

Medical system 200 includes an attachment device 230 for coupling accessory scope device 201 to primary scope device 102. Attachment device 230 may be removably coupled to primary scope device 102, e.g., at a proximal portion 111 of handle 104 on a side of handle 104 opposite of lever 112. Attachment device 230 may also be removably coupled to accessory scope device 201. Alternatively, attachment device 230 may be integral with handle 204.

In examples, attachment device 230 may include a channel 238 configured to receive handle 204 of accessory scope device 201. Channel 238 may be at least partially defined by a first coupler 234a and a second coupler 234b. A longitudinal axis of channel 238 may extend approximately parallel to a longitudinal axis of handle 104 when attachment device 230 is coupled to primary scope device 102. First and second couplers 234a, 234b may be used to secure handle 204 in place and to maintain a position of handle 204 relative to handle 104 (e.g., parallel to one another). As shown in FIG. 2A, a proximal portion 215 of handle 204 may be proximal of proximal portion 111 of handle 104 and a distal portion 217 of handle 204 is proximal of a distal portion 113 of handle 104. In examples, first and second couplers 234a, 234b may be clips or pincers, such that handle 204 of accessory scope device 201 may be clipped into channel 238.

Attachment device 230 may include an actuation assembly 236 with portions of actuation assembly 236 being contained within housing 232 of attachment device 230. Actuation assembly 236 may be coupled to actuation mechanism 209 (i.e., lever 212 and/or wheel 214) of accessory scope device 201 to control articulation of shaft 206. FIG. 2B shows a view of attachment device 230 that shows details of actuation assembly 236. For example, FIG. 2B may show a back side of attachment device 230 or attachment device with a portion of housing 232 removed.

Actuation assembly 236 may include a lever 244, a first arm 246a, a second arm 246b, and a third arm 246c movably coupled to one another for moving lever 212 (e.g., in an upward or downward direction). For example, third arm 246c may be coupled to lever 212. As discussed in further detail below, portions of lever 244 may have a bent shape, first arm 246a and third arm 246c may have a straight shape, and second arm 246b may have an arcuate shape. Lever 244, first arm 246a, second arm 246b, and third arm 246c may together form a linkage assembly, which may be external to primary scope device 102 and accessory scope device 201.

Lever 244 may be pivotable about a pivot point 263 in a direction M or a direction O. Pivot point 263 may include a pin, rivet, or similar structure that is directly or indirectly coupled to housing 232. Lever 244 may include a contact element 245 at a first end 255a (e.g., end 255a may be a free end). Contact element 245 may extend outside of housing 232, such that contact element 245 may be accessible to a hand of an operator. Contact element 245 may have a C or U shape. For example, an operator may place or rest a finger on contact element 245 and apply a force to contact element 245 to depress lever 244 in direction M. Actuation assembly 236 may include a tension spring 248 or other resilient member (e.g., coil spring, leaf spring, etc.) to urge or move lever 244 in direction O when, e.g., an operator stops applying a force to contact element 245. In other words, tension spring 248 may bias lever 244 to a relaxed/fully released configuration (i.e., a relaxed state). Alternatively, actuation assembly 236 may exclude tension spring 248, and contact element 245 may have a ring shape to allow an operator to move lever 244 in direction O using, e.g., the nail side of their finger. In another alternative, actuation assembly 236 may include tension spring 248, and contact element 245 may have a ring shape. In some examples, a bent shape of lever 244 may increase a mechanical advantage of lever 244 and/or provide a more ergonomic location of contact element 245.

Still referring to FIG. 2B, a second end 255b of lever 244 may be coupled to a first end 257a of first arm 246a via a first pin 242a. Although the term “pin” is used herein, it will be appreciated that the term “pin” may include rods, rivets, or the like. First pin 242a (and, thus, the junction of first arm 246a and lever 244) may be movable within a first slot 240a formed in housing 232. In some examples, first slot 240a may be approximately perpendicular to channel 238 and longitudinal axes of accessory scope device 201 and/or primary scope device 102. Slot 240a may constrain a motion of first pin 242a to be within slot 240a.

A second end 257b of first arm 246a may be coupled to a first end 259a of second arm 246b via a second pin 242b. Second pin 242b (and, thus, a junction between first arm 246a and second arm 246b) may be movable within a second slot 240b formed in housing 232. In some examples, second slot 240b may be approximately perpendicular to channel 238 and longitudinal axes of accessory scope device 201 and/or primary scope device 102. Second slot 240b may have a smaller length than first slot 240a, such that second pin 242b is constrained to a smaller range of motion than first pin 242a. Slot 240b may constrain a motion of second pin 242b to be within slot 240b.

As shown in FIG. 2B, slots 240a, 240b may be linear slots extending parallel to one another, and pins 242a, 242b may be movable within their respective slots 240a, 240b in directions A/B. Direction B may be opposite (e.g., approximately 180 degrees from) direction A. As shown in FIG. 2B, when first pin 242a moves in direction A, second pin 242b may move in direction B. When first pin 242a moves in direction B, second pin 242b may move in direction A.

A second end 259b of second arm 246b may be coupled to a first end 261a of third arm 246c via a hinge 249. A second end 261b of third arm 246c may be coupled (e.g., hingedly or fixedly coupled) to lever 212 of accessory scope device 201. Hinge 249 may be a living hinge (e.g., second arm 246b and third arm 246c may be integral with each other to form a single piece). Alternatively, hinge 249 may include a pin, and second arm 246b and third arm 246c may be separate pieces. Second end 261b of third arm 246c may be coupled to lever 212 by any suitable means (e.g., jaws, clips, straps, etc.). In some examples (in which, second end 261b may be integrally formed with 212 in configurations in which attachment device 230 is integrally formed with accessory scope device 201.

In use, movement of lever 244 in direction M about pivot point 263 may cause second end 255b of lever 244, along with first pin 242a and first end 257a of first arm 246a, to move in direction A, within first slot 240a. Movement of first end 257a of first arm 246a in direction A may cause second end 257b of first arm 246a, along with second pin 242b and first end 259a of second arm 246b, to move in direction B within second slot 240b. Movement of first end 259a of second arm 246b in direction B may cause second end 259b of second arm 246b and third arm 246c to move in at least partially a downward direction of FIG. 2B, which in turn moves lever 212 of accessory scope device 201 in the downward direction of FIG. 2B. Movement of lever 244 in direction O (or towards the relaxed/fully released configuration) may move lever 212 of accessory scope device 201 in an upward direction via the above steps in reverse.

With reference to FIG. 2A, during an exemplary medical procedure, an operator may couple accessory scope device 201 to primary scope device 102 using attachment device 230. The operator may grasp onto handle 104 of primary scope device 102, e.g., at grip portion 126, with a single hand. The operator may navigate shaft 106 of primary scope device 102 to a position proximate a target site (e.g., upper and deeper bronchioles), and may then insert shaft 206 of accessory scope device 201 into port 120 of primary scope device 102 and extend shaft 206 through the working lumen of primary scope device 102. Shaft 206 may exit the working lumen of primary scope device 102. The operator may use their thumb (or another finger) to control lever 112 to articulate shaft 106 (and portions of shaft 206 within shaft 106). The operator may use a finger (e.g., a finger of the same hand that controls lever 112) to control lever 244 of actuator assembly 236 to articulate shaft 206 independently of shaft 106. Articulation of shaft 206 may position shaft 206 at the target site (e.g., at a difficult to reach target site). The operator may actuate lever 112 and lever 212 (via lever 244) simultaneously or independently. The operator may then insert a medical instrument into port 220 and extend the instrument through a working lumen of shaft 206 and to the target site and/or deliver suction through shaft 206.

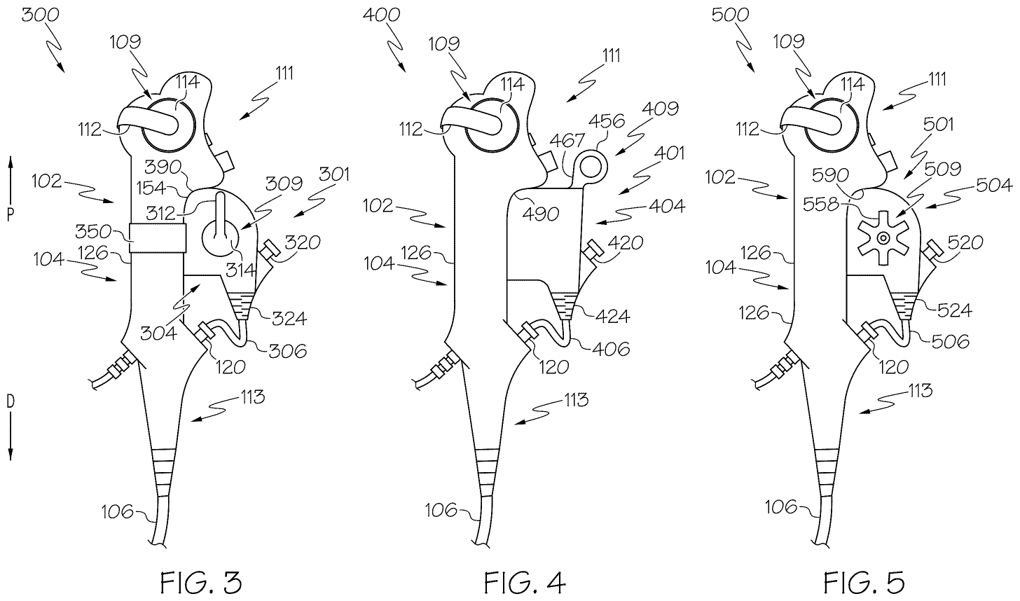

FIG. 3 illustrates a medical system 300 that includes primary scope device 102 coupled to an accessory scope device 301, which may have any of the features of accessory scope device 201, unless otherwise specified herein. Accessory scope device 301 may include a handle 304, a shaft 306, a stress relief portion 324, and a port 320 in communication with a working lumen of shaft 306. Handle 304 may include an actuation mechanism 309 having a lever 312 coupled to a wheel 314 (e.g., a cam wheel) to facilitate articulation of shaft 306.

Accessory scope device 301 may be sized and shaped to fit and/or rest against primary scope device 102. For example, handle 304 of accessory scope device 301 may have a compact shape. Handle 304 may include surfaces complementary to surfaces of handle 104 to facilitate coupling of handle 304 to handle 104. In other words, complementary surfaces of handle 104 and handle 304 may contact one another. For example, handle 304 may include surfaces complementary to surfaces of grip portion 126 of handle 104. In one example, handle 304 may have a flat/straight side that faces a flat/straight side of grip portion 126. Handle 304 may also include a curved surface 390 complementary to surfaces defining a nook 154 of handle 104.

Handle 304 may include a strap or clamp 350 (e.g., on a side of handle 304 opposite of port 320) for attaching accessory scope device 301 to primary scope 102. For example, clamp 350 may attach to grip portion 126 of handle 104. Alternatively, clamp 350 may attach to distal portion 113 of handle 104 (e.g., distal of port 120). It will be appreciated that clamp 350 may be substituted with any suitable attachment device described above (e.g., clips, pincers, etc.).

FIG. 4 illustrates a medical system 400 that includes primary scope device 102 coupled to an accessory scope device 401, which may have any of the features of accessory scope device 301 or 201, unless otherwise specified herein. Accessory scope device 401 may include a handle 404, a shaft 406, a stress relief portion 424, and a port 420 in communication with a working lumen of shaft 406. Similar to handle 304, handle 404 may include surfaces complementary to surfaces of handle 104. For example, handle 404 may include a curved surface 490 similar to curved surface 390. Handle 404 may have a shape that is similar to a rectangle having one or more curved corners (e.g., curved surface 490).

In this example, handle 404 includes an actuation mechanism 409 comprising a ring 456 and an arm 467 coupled to structures internal to a housing of handle 404 for controlling articulation of shaft 406. A housing of handle 404 may include an opening or slot, through which arm 467 may extend. An operator may move ring 456 relative to handle 404, which in turn may move arm 467 along the opening or slot formed on handle 404 to articulate shaft 406. Although not shown, handle 404 may include a clamp similar to clamp 350 or any other suitable attachment device for coupling handle 404 to handle 104.

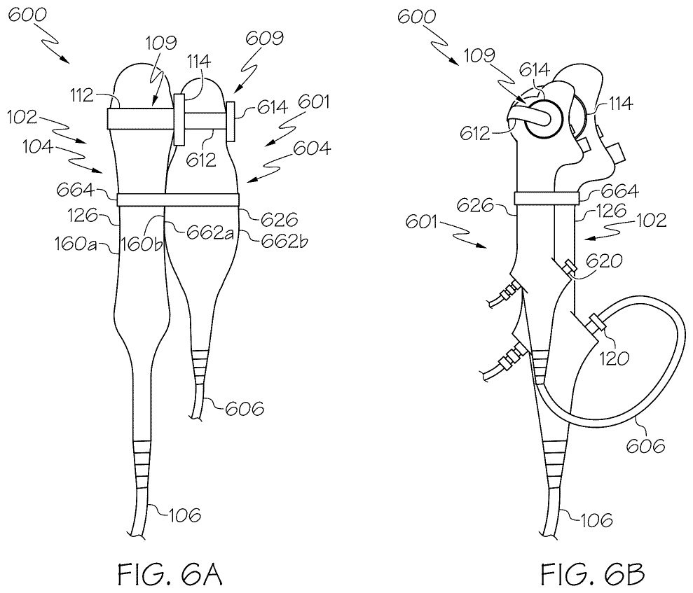

FIG. 5 illustrates a medical system 500 that includes primary scope device 102 coupled to an accessory scope device 501, which may have any of the features of accessory scope devices 201, 301, 401, unless otherwise specified herein. Accessory scope device 501 may include a handle 504, a shaft 506, a stress relief portion 524, and a port 520 in communication with a working lumen of shaft 506. Similar to handles 304, 404, handle 504 may include surfaces complementary to surfaces of handle 104. For example, handle 504 may include a curved surface 590 similar to curved surfaces 390, 490.

In this example, handle 504 includes an actuation mechanism 509 comprising a knob 558 coupled to structure internal to handle 504 for controlling articulation of shaft 506. An operator may rotate knob 558 relative to handle 504 to articulate shaft 506. Although not shown, handle 504 may include a clamp similar to clamp 350 or any other suitable attachment device for coupling handle 504 to handle 104.

FIGS. 6A and 6B illustrate a medical system 600 that includes primary scope device 102 coupled to an accessory scope device 601, which may have any of the features of accessory scope devices 201, 301, 401, 501, unless otherwise specified herein. FIG. 6A shows a back view of medical system 600 and FIG. 6B shows a side view of medical system 600. Accessory scope device 601 may include a handle 604, a shaft 606, and a port 620 in communication with a working lumen of shaft 606. Handle 604 may include an actuation mechanism 609 having a lever 612 coupled to a wheel 614 (e.g., a cam wheel) to facilitate articulation of shaft 606.

Primary scope device 102 and accessory scope device 601 may be coupled to one another via a band or strap 664. Strap 664 may wrap around portions of grip portions 126, 626 of handles 104, 604. Handle 604 may include surfaces complementary to surfaces of handle 104 to further facilitate coupling of accessory scope device 601 to primary scope device 102. For example, handle 604 may include convex side surfaces 662a, 662b complementary to concave side surfaces 160b, 160a of handle 104, respectively. As shown in FIG. 6A, convex side surface 662a of handle 604 may contact concave side surface 160b of handle 104. Alternatively, handle 604 may be placed on an opposite side of handle 104, such that convex side surface 662b of handle 604 may contact concave side surface 160a of handle 104.

Handle 604 may be positioned relative to handle 104, such that actuation mechanism 609 of accessory scope device 601 may be approximately aligned with actuation mechanism 109 of primary scope device 102. In use, an operator may grasp onto both handles 104, 604 with one hand, and may control levers 112, 612 using their thumb or other finger(s). Alignment of levers 112, 612 may allow the operator to more easily move their thumb back and forth from lever 112 to articulate shaft 106 to lever 116 to articulate shaft 606. In some examples, an operator may contact levers 112, 612 simultaneously to actuate levers 112, 612 at the same time.

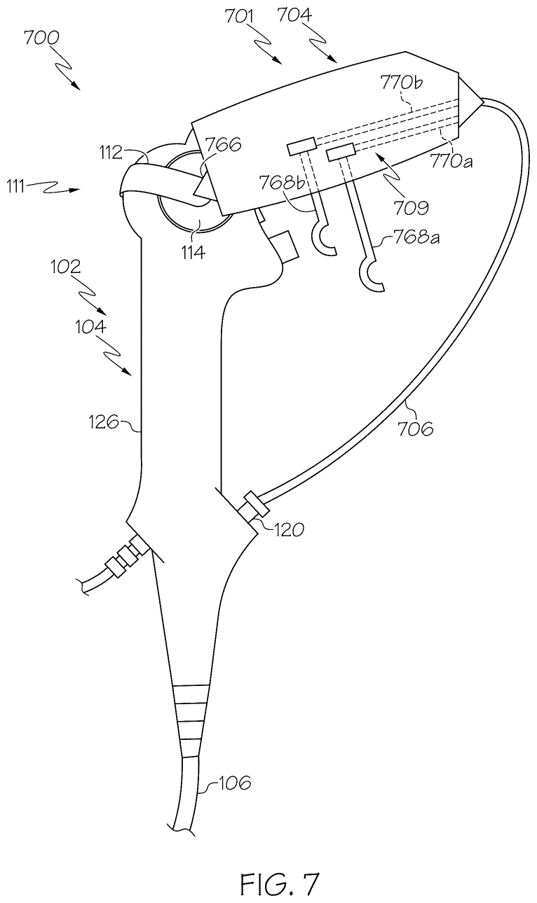

FIG. 7 illustrates a medical system 700 that includes primary scope device 102 coupled to an accessory scope device 701. Accessory scope device 701 may include a handle 704 having any of the features of handles 204, 304, 404, 504, 604 and a shaft 706 having any of the features of shafts 206, 306, 406, 506, 606, unless otherwise specified herein.

Handle 704 may include a clip 766 for attaching handle 704 to handle 104 of primary scope device 102. Clip 766 may attach to proximal portion 111 of handle 104, such that handle 704 may be on a side of handle 104 opposite of lever 112. For example, clip 766 may attach to wheel 114 in such a manner that wheel 114 may move (rotate) relative to clip 766. Handle 704 may extend substantially perpendicular to a longitudinal axis of handle 104 when handle 704 and handle 104 are coupled to one another. Although handle 704 is described as including clip 766, it will be appreciated that handle 704 may include any suitable attachment device, such as those described above.

Handle 704 may include an actuation mechanism 709 having a first trigger 768a and a second trigger 768b coupled to structure internal to a housing of handle 704 for controlling articulation of shaft 706. For example, first trigger 768a may be coupled to a first pull wire 770a and second trigger 768b may be coupled to a second pull wire 770b. First and second pull wires 770a, 770b may extend through handle 704 and through shaft 706 to facilitate articulation of shaft 706. First and second triggers 768a, 768b may be movable within respective slots or openings of handle 704. For example, first and second triggers 768a, 768b may be movable in proximal/distal directions relative to handle 704. In some examples, triggers 768a, 768b may be linearly movable (e.g., may be sliders). In other examples, triggers 768a, 768b may be pivotably movable.

Movement of first trigger 768a in a first direction (e.g., a proximal direction) may move pull wire 770a in the same direction (e.g., a proximal direction). Similarly, movement of second trigger 768b in a second direction (e.g., a distal direction) may move second pull wire 770b in the same direction (e.g., a distal direction). In some examples, first trigger 768a and second trigger 768b may be simultaneously movable in opposite directions (one moved proximally and one moved distally) to articulate a distal end of shaft 706 in a first direction. In some examples, triggers 768a and/or 768b may include loops to facilitate proximal and distal movement of triggers 768a and/or 768b. Moving first trigger 768a and second trigger 768b in respective opposite directions may articulate the distal end of shaft 706 in a second direction, opposite to the first direction. Alternatively, only one of first trigger 768a and second trigger 768b may be moved at a time. First trigger 768a and second trigger 768b may thus be used to articulate the distal end of shaft 706 in two directions. In examples, handle 704 may include additional triggers that may be used to articulate the distal end of shaft 706 in additional directions.

In use, an operator may grasp onto handle 104, e.g., at grip portion 126, with one hand. The operator may use their thumb to control lever 112 and other fingers of the same hand to control first and second triggers 768a, 768b. For example, the operator's fingers may wrap around grip portion 126 to actuate one or more of triggers 768a, 768b. The operator may be able to simultaneously actuate control lever 112 and first and second triggers 768a, 768b.

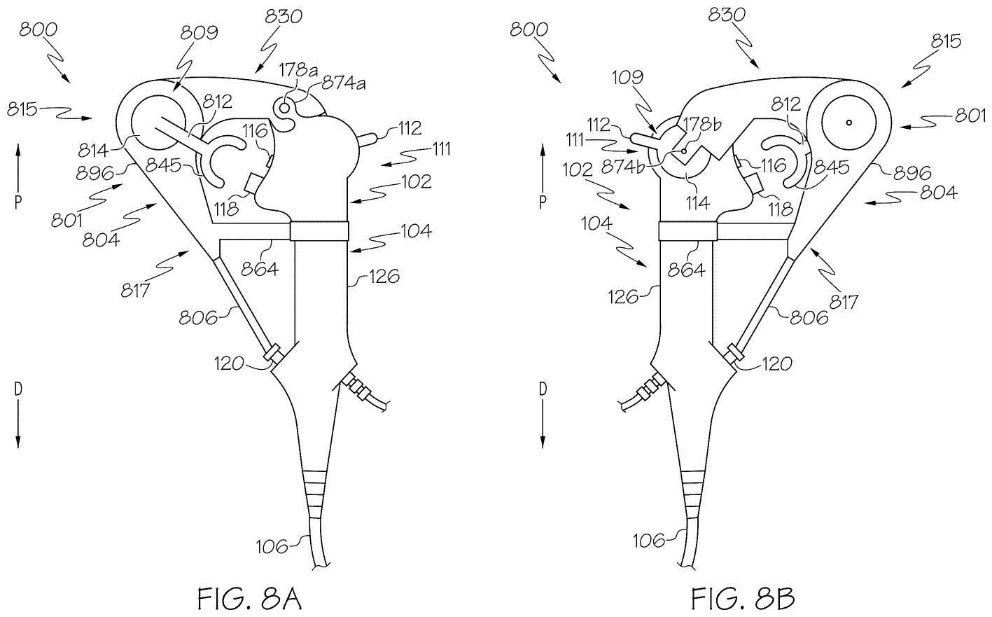

FIGS. 8A and 8B illustrate side views of a medical system 800 that includes primary scope device 102 coupled to an accessory scope device 801. Accessory scope device 801 may include a handle 804 having any of the features of handles 204, 304, 404, 504, 604, 704 and a shaft 806 having any of the features of shafts 206, 306, 406, 506, 606, 706, unless otherwise specified herein.

Handle 804 may include an attachment portion 830 and a strap 864 or other attaching mechanism for attaching and/or securing handle 804 to handle 104. Portions of attachment portion 830 and/or strap 864 may extend substantially perpendicular to a longitudinal axis of handle 104. Although a strap 864 is shown, it will be appreciated that, instead, handle 804 may include a clamp, pincer, or deformable housing for receiving a portion of handle 104 (such as, for example, grip portion 126). Strap 864 may be at a distal portion 817 (e.g., near or at a distal end) of a handle body 896 of handle 804.

Attachment portion 830 may, for example, attach and/or secure handle 804 on a side of handle 104 opposite of lever 112 (e.g., on the same side as button 116 and valve 118). Attachment portion 830 may be positioned at a proximal portion 815 of handle body 896. Attachment portion 830 may include openings, recesses, or holes 874a, 874b for receiving pins 178a, 178b (or other protruding portions) of handle 104, respectively, to secure attachment portion 830 to proximal portion 111 of handle 104. As shown in FIG. 8B, pin 178b may extend from wheel 114 of handle 104. Pin 178a may extend from an opposite side of handle 104 at a corresponding location to pin 178b. Alternatively, attachment portion 830 may include pins or other protrusions corresponding to openings, recesses, or holes of handle 104. For example, pins 178a, 178b may instead be openings, recesses, or holes and openings, recesses, or holes 874a, 874b may instead be pins.

When attachment portion 830 is coupled to handle 104, a distance from proximal portion 815 of handle body 896 to handle 104 may be greater than a distance from distal portion 817 of handle body 896 to handle 104, such that handle body 896 may be angled relative to handle 104, with handle body 896 extending away from handle 104 in a proximal direction. Handle 804, including attachment portion 830, may have an overall shape that is similar to a “V” or a “U.” Proximal portion 815 of handle body 896 having a lever 812 (discussed in further detail below) may be a vertex of the “V” or “U” shape, with attachment portion 830 and distal portion 817 extending away from proximal portion 815 of handle body 896.

Handle 804 may include an actuation mechanism 809 having lever 812 coupled to wheel 814 (e.g., a cam wheel) to facilitate articulation of shaft 806. In this example, lever 812 may extend downward from wheel 814 and toward handle 104, e.g., toward button 116 and valve 118, when attachment portion 830 is coupled to handle 104. In use, lever 812 may extend downward toward an operator. Lever 812 may include a contact element 845 having a C or U shape facing handle 104 (or facing the operator during use of system 800). Alternatively, contact element 845 may be a ring.

In use, an operator may grasp onto handle 104, e.g., at grip portion 126, with one hand. The operator may use their thumb to control lever 112 to articulate shaft 106 and a finger (e.g., an index finger) of the same hand to rotate lever 812 to articulate shaft 806.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A medical system comprising:
a first medical device including a handle and a shaft extending distally from the handle;
a second medical device including a handle having an actuator and a shaft extending distally from the handle, wherein the actuator is configured to deflect the shaft of the second medical device; and
an attachment device configured to removably couple the first medical device to the second medical device, the attachment device comprising a linkage that is configured to be coupled to the actuator, wherein the linkage is external to the handle of the second medical device, wherein the linkage includes a lever, and wherein movement of the lever is configured to articulate the actuator of the second medical device.

2. The medical system of claim 1, wherein the attachment device includes a housing that is movably coupled to one or more elements of the linkage, wherein the attachment device further comprises:
a channel for receiving the second medical device.

3. The medical system of claim 1, wherein the attachment device is removably coupled to the first medical device and the second medical device.

4. The medical system of claim 1, wherein the attachment device is configured such that an actuator of the first medical device and the lever of the attachment device are actuatable by a single hand of a user.

5. The medical system of claim 1, wherein the linkage further includes a first arm, a second arm, and a third arm movably coupled to one another.

6. The medical system of claim 5, wherein a first end of the lever is free and a second end of the lever is coupled to a first end of the first arm, wherein a second end of the first arm is coupled to a first end of the second arm, wherein a second end of the second arm is coupled to a first end of the third arm, and wherein a second end of the third arm is coupled to the actuator of the second medical device.

7. The medical system of claim 5, wherein the third arm has an arcuate shape.

8. The medical system of claim 6, wherein the second end of the lever is coupled to the first end of the first arm via a first pin movable within a first slot of a housing of the attachment device, and wherein the second end of the first arm is coupled to the first end of the second arm via a second pin movable within a second slot of the housing.

9. The medical system of claim 8, wherein the first slot and the second slot extend parallel to one another.

10. The medical system of claim 6, wherein the second end of the second arm is coupled to the first end of the third arm via a hinge.

11. The medical system of claim 10, wherein the hinge is a living hinge.

12. The medical system of claim 2, wherein the lever includes a contact element positioned outside of the housing of the attachment device.

13. The medical system of claim 12, wherein the contact element has a C or U shape.

14. The medical system of claim 12, wherein the contact element has a ring shape.

15. The medical system of claim 1, wherein the linkage further includes a spring coupled to the lever, and wherein the spring is configured to bias the lever to a relaxed state.

16. A medical system comprising:
a first medical device including a handle coupled to a shaft and an actuator for deflecting the shaft of the first medical device;
a second medical device including a handle, a shaft coupled to the handle, and an actuator for deflecting the shaft of the second medical device; and
one or more attachment devices coupling the handle of the second medical device to a proximal portion of the handle of the first medical device, such that the actuator of the second medical device is positioned on a first side of the handle of the first medical device, wherein the actuator of the first medical device is positioned on a second side of the handle of the first medical device, wherein the first side is opposite to the second side, and wherein the actuator of the second medical device and the actuator of the first medical device are actuatable by a single hand of a user.

17. The medical system of claim 16, wherein the one or more attachment devices couple to a wheel of the actuator of the first medical device.

18. The medical system of claim 16, wherein the actuator is a lever angled towards the handle of the first medical device in a configuration in which the one or more attachment devices couple the handle of the second medical device to the handle of the first medical device.

19. A medical system comprising:
a first medical device including a handle coupled to a shaft; and
a second medical device including:
a shaft; and
a handle coupled to the shaft, wherein the handle includes:
a handle body;
an actuation mechanism for deflecting the shaft of the second medical device; and
an attachment device configured to couple the handle body of the second medical device to the handle of the first medical device, wherein the handle has a substantially “V” shape.

20. The medical system of claim 19, wherein a distal portion of the handle body and the attachment device extend away from a proximal portion of the handle body, and wherein the proximal portion of the handle body includes the actuation mechanism.
